(19) United States
(12) Patent Application Publication
(10) Pub. No.: US 2015/0245570 A1
(43) Pub. Date: Sep. 3, 2015

(54) REVERSE SYNTHESIS OF BREEDING LINES

(71) Applicant: RIJK ZWAAN ZAADTEELT EN ZAADHANDEL B.V., KX De Lier (NL)

(72) Inventors: Arie VOGELAAR, ZC Dordrecht (NL); Rudolf VERHOEF, GK Naaldwijk (NL); Johannes Wilhelmus SCHUT, BC Wouw (NL); Robert Hélène Ghislain DIRKS, GL Oudenbosch (NL)

(21) Appl. No.: 14/709,947

(22) Filed: May 12, 2015

Related U.S. Application Data
(63) Continuation-in-part of application No. PCT/EP2013/073976, filed on Nov. 15, 2013.

(30) Foreign Application Priority Data
Nov. 15, 2012 (EP) 12192813.9

(51) Int. Cl. A01H 1/04

(52) U.S. Cl. CPC A01H 1/04

(57) ABSTRACT

The invention relates to a method for producing parental lines for a hybrid organism. This method is based on the segregation of individual alleles in the spores produced by a desired plant and/or in the progeny derived from the self-pollination of that desired plant, and on the subsequent identification of suitable progeny plants in one generation, or in a limited number of inbred cycles. The invention further relates to a method for reconstructing a partially heterozygous starting organism.

### RELATED APPLICATIONS AND INCORPORATION BY REFERENCE

This application is a continuation-in-part application of international patent application Serial No. PCT/EP2013/073976 filed Nov. 15, 2013, which published as PCT Publication No. WO 2014/076249 on May 22, 2014, which claims benefit of European patent application Serial No. 12192813.9 filed Nov. 15, 2012.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

This invention relates to a method for producing parental lines for a hybrid organism.

### BACKGROUND OF THE INVENTION

Plant breeding has the objective to produce improved crop varieties based on available genetic variation. In traditional plant breeding, the person skilled in the art selects individual plants that are supposed to possess genes or traits of interest and subsequently either crosses are made in order to combine the gene(s) of the desired trait(s) with genes that encode other useful characteristics, and/or self-pollinations are made in order to try to obtain true breeding lines, which means that those plants produce only progeny like themselves.

Since the early days of plant breeding, tremendous technical progress has been made to facilitate selection, such as the invention of doubled haploid techniques either by regeneration of plants derived from haploid egg cells (gynogenesis) or regeneration of plants derived from microspores (androgenesis). Such plants are generally haploid, unless spontaneous diploidisation had occurred during the procedure. Chemical compounds that interfere with mitosis (such as colchicin) may also be used to double the genome of haploid plants. Plants derived from such Doubled Haploid technology are completely homozygous and breed true.

“True breeding” refers to a situation in which a pair of parental lines consistently produces offspring with particular phenotypic characteristics, such that the next generation consists entirely of phenotypically homogenous individuals.

In many crop species it is desired to make F1 hybrid varieties. Such varieties are usually based on the crossing of two true breeding lines that may genetically complement each other, but epistasis causing hybrid vigour may also occur. Often this complementarity of genotypes or epistatic interactions between genomes of genetically different parental lines, in the hybrid results in a considerable improvement of e.g. growth characteristics, yield or adaptation to environmental stresses as compared to the individual parental lines. Such enhancement of yield or strength is generally referred to as heterosis or hybrid vigour. Another term that relates to heterosis is “combining ability”. Combining ability is the phenomenon that some true breeding lines, when crossed to each other, complement each other in desired traits or enhance some traits, or in the opposite case (bad combining ability) may result in an F1 hybrid that is not suitable or better than either of the individual parental lines. Very often, negative heterosis or a lack of heterosis can be explained by negative epistasic effects. In general, so-called test crosses are performed between putative parental lines to investigate the performance of the resulting offspring.

It may thus well be the case that an F1 hybrid variety is composed of two genetically very different true breeding parental lines and that (as a consequence of bringing together complementary alleles) it performs better than the parental lines. Thus, for several genes in the F1 hybrid the allelic status at a given locus may be dissimilar in the original parental lines, and for those loci the plant will thus be heterozygous.

It is possible that, by chance, in the progeny of random crosses between parents that are not fully homozygous or even substantially heterozygous, an individual plant is encountered, carrying a highly desirable set of allelic combinations. This individual plant, being heterozygous and unique, may be propagated vegetatively if suitable in vitro and/or in vivo methods are available. This is in fact a well-established procedure in potato, where vegetative propagation occurs via natural tubers. In ornamental and tree breeding similar situations occur. In most of the field crops and greenhouse crops, however, breeding is carried out on the basis of true breeding lines and combining ability testing.

The traditional method of parental line development does not allow for the selection of lines that, when crossed, would result in a predictable phenotype that had already earlier been identified in a randomly segregating population. When a plant with a desirable phenotype would be self-pollinated, segregation of loci and their corresponding alleles will take place either independently and/or linked, depending on the relative genomic position of the loci. The former is valid e.g. in cases where loci/alleles are located on different chromosomes. The latter is valid in cases where loci/alleles are located on the same chromosome (i.e. they are genetically linked to some extent, as a result of which their segregation depends on the recombination breakpoint(s) within the chromosomal region). In classical breeding by means of phenotypic selection it is therefore very unlikely that lines can be developed that, when crossed, will reconstruct the original desirable heterozygous individual (herein referred to as the “starting hybrid”, “starting organism” or the “partially heterozygous starting organism”). An additional drawback of this type of approach is that the selected plants from the progeny of the self-pollinated starting organism still remain heterozygous for a considerable number of regions of the genome. This implies that when parental plants yielding a successful test cross (with progeny that closely resembles the original starting organism) are propagated through self-pollination, the resulting progeny plants are not genetically identical. They will still segregate for a number of chromosomal regions, which may result in the undesired segregation for phenotypic properties. This deficiency hampers the development of genetically uniform hybrid seed lots.

Previously, a method (referred to as Reverse Breeding) has been described in WO2003/017753, that allows the construction of parental lines from a single desirable plant with an unknown genetic constitution, that, when crossed, may exactly or partly reconstitute the said desirable plant in a reproducible fashion and on a large scale, so that in fact an individual plant may be converted to a new variety by means of the creation of suitable parental lines that genetically complement each other. Such parental lines are traditional in a genetic sense because they are true breeding, and the F1 hybrid is in a genetic sense also traditional because it has been derived from a cross between two true breeding lines. However, the breeding process itself is fundamentally reversed. Instead of analysing the “combining suitability” of two parental lines to result in a superior hybrid, a plant that is judged to be superior is used as the starting material for the development of suitable corresponding breeding lines, that can be subsequently used for the consistent and large-scale production of that superior starting organism.

The Reverse Breeding procedure comprises a method for inhibiting recombination during meiosis, followed by a doubled haploid step. By this method, surprisingly low numbers of doubled haploids produced from a donor plant in which recombination is suppressed (e.g. by transgenic methods or by chemical inhibitors) are sufficient to allow the identification of genetically pure lines that are suitable for the conversion of a desired partially heterozygous starting plant into a desirable F1 hybrid with essentially the same phenotypic characteristics, or at least one of the phenotypic characteristics.

In the research that led to the present invention, another method has been developed that allows the creation of suitable parental lines from a partially heterozygous starting organism, however without the necessity to suppress meiotic recombination, and for which doubled haploids are not a prerequisite, although they may be a useful addition.

The new method of this invention is based on the segregation of individual alleles in the spores produced by a desired plant (herein referred to as the “starting hybrid”, “starting organism” or “partially heterozygous starting organism”) and/or in the progeny derived from the self-pollination of that desired plant, and on the subsequent identification of suitable progeny plants in one generation (with the aid of doubled haploid technology), or in a limited number of inbred cycles. In the context of this invention, meiotic recombination is not suppressed or prevented in any of the method steps.

In one embodiment, which is illustrated in Example 2, the current invention relates to a method for producing parental lines for a hybrid organism, comprising:
a) defining a set of genetic markers that are present in a heterozygous form (H) in a partially heterozygous starting organism;
b) producing doubled haploid lines from spores of the starting organism;
c) genetically characterising the doubled haploid lines thus obtained for the said set of genetic markers to determine whether they are present in a first homozygous form (A) or in a second homozygous form (B);
d) selecting at least one pair of doubled haploid lines that have complementary alleles for at least a subset of the genetic markers, wherein each member of the pair is suitable as a parental line for a hybrid organism.

The first and second homozygous form A and B referred to in step c) are complementary to each other, because they are alleles of co-dominant markers, whose simultaneous presence in an organism is scored as H (heterozygous).

In one embodiment of the claimed invention, the genetic markers from the set of genetic markers are distributed essentially evenly across the genome and/or across the genetic map of the organism. This ensures that essentially all chromosomes and essentially all chromosomal regions can be investigated by means of the set of genetic markers, which provides an advantage in the context of this invention.

The wording “a subset of the genetic markers” in step d) refers to a subset of the genetic markers from the set of genetic markers that had been defined in step a). The subset may comprise as few as one or two markers, but suitably, the subset of genetic markers referred to in step d) comprises a majority of the genetic markers from the set of genetic markers, or essentially all genetic markers from the set of genetic markers, or all genetic markers from the set of genetic markers.

In one embodiment, the doubled haploid lines of step b) are produced in vitro by means of gynogenesis or androgenesis, followed by diploidisation, in particular by spontaneous or chemical-induced diploidisation. Chemical induction of diploidisation can e.g. be achieved by treatment with compounds such as (but not restricted to) colchicine, oryzalin or trifluralin.

In another embodiment, the doubled haploid lines of step b) are produced in vivo, in particular by means of a haploid-inducer system. Haploid inducer systems have been described in various plant species. In interspecific crosses, loss of the genome of one of the parents has often been observed, such as in the cross between wheat and pearl millet, between barley and Hordeum bulbosum, and between tobacco (Nicotiana tabacum) and Nicotiana africana. In one embodiment, the female parent in a cross thus belongs to a different species than the male parent in the cross.

The female parent in a cross can also be a transgenic plant that comprises a heterologous transgene expression cassette, the expression cassette comprising a promoter operably linked to a polynucleotide encoding a recombinantly altered CENH3, CENPC, MIS12, NDC80 or NUF2 polypeptide, and having a corresponding inactivated endogenous CENH3, CENPC, MIS12, NDC80 or NUF2 gene. This embodiment has been first described in Arabidopsis thaliana (Maruthachalam Ravi & Simon W. L. Chan; Haploid plants produced by centromere-mediated genome elimination; Nature 464 (2010), 615-619; US-2011/0083202; WO2011/044132), and it is broadly applicable in plants.

In one embodiment, doubled haploid lines (DHs) are thus produced from the starting organism. Those DHs that are most complementary to each other (within the population that had been generated) with respect to a set of selected genetic markers are selected and crossed with each other as parental lines for a hybrid. Because recombination proceeds normally (in contrast to the procedure of Reverse Breeding), it is surprising that DH lines can be found that are sufficiently complementary to allow the re-synthesis of the original starting organism. This approach allows the rapid identification of suitable DHs that, when crossed, may give rise to a hybrid that is genetically essentially identical to the starting organism from which the DHs had been derived, or at least partially identical to this hybrid, with respect to the genetic marker profile. In one embodiment, the new hybrid displays all, nearly all or essentially all of the phenotypic characteristics for which the starting organism had been selected. In other embodiments, however, fewer phenotypic characteristics of the starting organism may be needed and thus the new hybrid can also comprise at least one of those phenotypic characteristics. Based on the marker profiles of the newly developed parental lines any desirable combination can be made ranging from a hybrid that has an exact copy of the marker profile of the original starting organism to a hybrid that has retained only one or a few markers that correspond with desirable characteristics.

This method is generally applicable in all species for which DHs can be made.

When the method of the invention is carried out by means of DH technology, as described above, the resulting DHs may be crossed to each other in a full- or half-diallele fashion. In rare cases meiotic recombination may incidentally not take place in at least some of the chromosomes, or even in all chromosomes, even though it had not been deliberately suppressed or prevented by the person carrying out the experiment. This may lead to the production of DHs with unrecombined chromosomes. If such surprising events can be detected among the DH population derived from the starting organism, an extreme scenario is encountered in which it becomes possible to exactly reconstruct the starting organism, with 100% genetic identity, if DHs can be identified that have fully complementary sets of unrecombined chromosomes. If meiotic recombination does occur in at least one of the chromosomes of the starting organism, then the genotype of the starting organism can usually not be reconstructed for 100% by crossing of DHs, but its genotype is approximated. In the latter case, the phenotypic characteristics of the starting organism may nevertheless be entirely or essentially be present in the hybrid organism resulting from the cross between selected DHs.

In the context of this invention, “complementary” refers to the allelic form of genetic markers that can be co-dominantly scored in the selected offspring plants, and especially of genetic markers that are present in a heterozygous state in the starting organism. The terminology used in this context is A for homozygous allele 1, B for homozygous allele 2, and H for the heterozygous presence of both A and B. The allelic forms A and B are complementary to each other for the purpose of this invention, because crossing of a plant having allelic form A to another plant having allelic form B for the same locus will give rise to an F1 generation in which both A and B will be present in a heterozygous form, i.e. the locus will be scored as H. To reconstruct a hybrid or at least partially heterozygous starting organism as completely as possible, one should ideally identify progeny plants that are pairwise complementary for essentially all genetic markers (i.e. essentially all chromosome regions) for which the starting organism was heterozygous, because these genetic markers and chromosome regions are expected to contribute to the superior phenotypic performance of the starting organism.

In another embodiment, which is illustrated in Example 1, the current invention relates to a method for producing parental lines for a hybrid organism, comprising:
a) defining a set of genetic markers that are present in a heterozygous form (H) in a partially heterozygous starting organism;
b) producing at least one further generation from the starting organism by self-pollination;
c) selecting at least one pair of progeny organisms in which at least one genetic marker from the set is present in a complementary homozygous form (B vs. A, or A vs. B);
d) optionally repeating steps b) and c) until at least one pair of progeny organisms that have complementary alleles for at least a subset of the genetic markers has been selected as parental lines for a hybrid.

Step d) is optional in this method, because it does not need to be carried out if the at least one pair of progeny organisms that have complementary alleles for at least a subset of the genetic markers had already been selected as parental lines for a hybrid in the first inbreeding generation (F2).

The wording “a subset of the genetic markers” in step d) refers to a subset of the genetic markers from the set of genetic markers that had been defined in step a). Suitably, the subset of genetic markers referred to in step d) comprises a majority of the genetic markers from the set of genetic markers, or essentially all genetic markers from the set of genetic markers, or all genetic markers from the set of genetic markers.

In this embodiment, doubled haploids are not used for hybrid re-synthesis, but instead repeated cycles of inbreeding and analyses of genetic markers are carried out to eventually lead to recombinant inbred lines homozygous for the traits of interest, and among which lines can be identified that are dissimilar to each other (i.e. complementary) with respect to a large number of informative genetic markers. This method is generally applicable in all crops for which genetic marker analysis can be carried out.

Apart from essentially reconstructing the genetic marker profile (and preferably also essentially the desirable phenotypic characteristics) of the partially heterozygous starting organism, it is also possible during the reconstruction to remove or repair sub-optimal or undesirable phenotypic characteristics that were present in the starting organism. If the undesirable trait was present in a heterozygous state in the starting organism, the person skilled in the art knows that the F2 population will segregate in a 1/2/1 ratio for this trait, if it is controlled by a single locus. If the undesirable trait is dominant, then 25% of the F2 organisms will not phenotypically show the trait. If organisms are selected from this subset of the F2 population for use in the further steps of the method of the invention, the said undesirable trait will be absent from the parental lines and the hybrid organism that ultimately result from the method. Suitably, more than one undesirable trait can be removed in this manner. Preferably, the individuals are scored for genetic markers that are genetically linked to said undesirable trait or traits, such that efficient selection for the absence (or presence) of said undesirable trait or traits is possible among the F2 and subsequent generations, without a need to phenotypically assess the presence or absence of the undesirable trait or traits in every single individual and generation.

The two alternative methods outlined above (i.e. the method of the invention using DHs or inbreeding, respectively) may also be combined with each other, as illustrated in Example 3. In this combined approach one of the parental lines may be obtained using the method of the invention using DH technology, whereas the other parental line may be obtained using the method of the invention using inbreeding. The approach illustrated in Example 3 thus combines the power of each of the two main embodiments of the method of the invention: the creation of DHs from the starting organism leads to immediate fixation of all genetic markers in one of the two available allelic forms, whereas the use of repeated inbreeding of the same starting organism postpones genetic fixation by leaving many of the informative markers in a heterozygous state, which inherently enables the fixation of either of the two allelic forms in the next or subsequent generations. In this combined approach one thus rapidly fixes all genetic markers in one candidate parental line (through creation of DHs from the starting organism), and subsequently one screens among the progeny derived from selfing of the starting organism for a second parental line, that has maximal complementarity at the level of the selected set of informative genetic markers.

In one embodiment of the claimed invention, the genetic markers from the set of genetic markers are distributed essentially evenly across the genome and/or across the genetic map of the organism. This ensures that essentially all chromosomes and essentially all chromosomal regions can be investigated by means of the set of genetic markers, which provides an advantage in the context of this invention.

A typical experimental set-up to carry out the method of the invention consists for example essentially of three steps: (1) the determination of a (preferably large) set of informative genetic markers, optionally (2) the construction of a genetic map based on the selected genetic markers, and (3) the selection from a preferably large population of plants with complementary genotypes. Other experimental set-ups are also possible. Plants are considered to have “complementary genotypes” when they have complementary alleles for at least a subset of the set of genetic markers, preferably for a majority of the genetic markers of the set of genetic markers, or for essentially all genetic markers of the set of genetic markers, or for all genetic markers of the set of genetic markers.

Step 1: the determination of a set of informative genetic markers starts with the genetic analysis of either the starting organism itself or of a limited number of offspring plants that originate from the self-pollination of the starting organism. For this analysis either a set of randomly chosen co-dominant genetic markers is used, or a set of well-defined co-dominant markers with a known position in the genome and/or on the genetic map, such that all or most chromosomes of the species under study are covered with genetic markers that are preferably well distributed across each of the individual chromosomes (or linkage groups). This step provides knowledge about the amount of genetic variation that exists within the preferred plant, and subsequently about the number of genetic markers that are needed to efficiently and effectively analyse the genetic configuration of the complete population or of a subset of the population derived from the self-pollination of the starting organism. The use of co-dominant genetic markers is essential, because this type of marker enables the differentiation of both alleles at a given locus, and hence allows the recognition of all three genetic configurations at a given locus, whereby A=homozygous allele 1; B=homozygous allele 2 and H=heterozygous.

Step 2: the construction of a genetic map based on the selected genetic markers is performed by analysing the segregation of the selected genetic markers in the complete population or in a subset of the population derived from the self-pollination of the starting organism, and hence determining the recombination fractions of the genetic markers. These recombination fractions are relatively easy to determine if the linkage phase (coupling phase=markers are located on the same chromosome, or repulsion phase=markers are located on opposite chromosomes) of the markers in the parents is known. In this case it is easy to determine which gametes are recombinant and which ones are non-recombinant. In a normal F2 population based on two true breeding lines, the linkage phase of the genetic markers is known. However, in case of a single starting organism the linkage phase of the genetic markers is not known in advance. Therefore, one has to correct for this and infer the parental linkage phase, as the number of recombinants is expected to be smaller than the number of non-recombinants. Step 2 is optional, and does not need to be performed if a suitable genetic map and/or a genome sequence is already available.

Step 3: the selection of plants with complementary genotypes is performed either on the basis of (1) the allelic forms detected in each plant for the individual genetic markers, whereby the best pairs of complementary plants are as dissimilar as possible to each other with respect to the tested genetic markers (i.e. they have as many genetic markers as possible that score A in one of the selected plants and that score B in the complementary plant of the pair, and more importantly, especially in the early inbreeding generations, they have as few genetic markers as possible that score the same homozygous allelic state (A or B) in both plants of the pair), or (2) essentially the same as (1), but combined with the knowledge of the calculated map positions and linkage phase of the genetic markers used (which allows the targeted selection of genetic markers that essentially cover the complete genome or the complete genetic map).

By using this information, it is possible to estimate the probabilities for each set of plants to generate complementary homozygous inbred lines by further inbreeding and selection, as well as the optimal size of the population. In the latter case the visualisation of the genotypes of the individual plants enables to determine the number of genomic regions that are fixed for either one of the alleles, and those that are still heterozygous. If a genomic regions is heterozygous both allelic forms of genetic markers in that region are still present, and fixation may still proceed in either direction (A or B) in the next generation, or in subsequent generations.

In order to visualize the different genotypes and marker scores of the individual plants various software packages are available, either publicly (see, for example, Graphical GenoTypes, http://www.wageningenur.nl/nl/show/Graphical-GenoTypes-transform-molecular-data-to-colorful-chromosome-drawings.htm and Van Berloo, Journal of Heredity, 1999 (Vol. 90: 328-329) or proprietary (see, for example, Genome Typer, Keygene N.V.). In most cases the minimal input of such software packages is a genetic map (e.g. a dm1-file) and a genotype file (e.g. a loc-file).

The procedure outlined above may be reiterated as many times as necessary, until genetically complementary plants have been identified. Suitably, this may occur in the F2, F3, F4, F5, F6, or in subsequent generations. FIG. 1 illustrates the progressive fixation of genetic markers in F2, F3, F4, F5 and F6 generations, derived from a partially heterozygous starting organism through repeated inbreeding. In each panel the marker scores for a number of individuals are presented. Each horizontal line corresponds to a different individual from a certain generation, and from left to right the different linkage groups are represented. In this dataset the various individuals can be compared to each other based on their overall complementarity with respect to the set of genetic markers, and sets of individuals that are at least partially complementary can be identified. The chance of identifying a higher degree of complementarity increases in every generation, as more of the genetic markers become genetically fixed in one of their allelic forms (A or B). As long as a chromosome region is heterozygous (which may be assessed by scoring the genetic markers it comprises) it retains the potential to become fixed in either of the two allelic forms in the next generation or in further generations. When selecting genetically complementary plants, there is no need to limit the search to individuals from the same generation. One may e.g. identify a largely homozygous individual in the F3 generation, and find a suitable crossing partner for this individual in the F5 generation derived from e.g. another F3 individual through two successive inbreeding cycles.

Whenever partially complementary plants have been identified that are genetically complementary for a subset of the informative genetic markers, but that are heterozygous for the remaining informative genetic markers, these plants are self-pollinated and steps 1-3 are repeated, with the aim of identifying offspring plants that are complementary to each other with respect to the genetic markers that were still present in a heterozygous state in the previous generation. In this way, all genomic regions that were still present in a heterozygous state in each selected plant (as experimentally revealed by the heterozygous presence of at least one genetic marker in such genomic regions) will ultimately become fixed for allelic form A or for allelic form B for essentially all of the informative genetic markers. When complementary lines are eventually crossed to give rise to a hybrid plant, this hybrid plant will genetically (and preferably also phenotypically) resemble the starting plant, especially with respect to the investigated molecular markers.

The genome of the starting organism can be compared to a closed box: by self-pollinating this starting organism, its genome is shuffled due to meiotic recombination, but no new alleles are allowed to enter the population. Once an allelic form is present in a homozygous state, it will remain fixed during subsequent generations of selfing, and it cannot change back into the other allelic form (unless it spontaneously mutates to give rise to the other allelic form, but such an event is expected to be very rare). The method of the invention ideally strives towards a state wherein all informative genetic markers are present in a homozygous state in individual progeny plants, and wherein at least one pair of individual progeny plants can be identified and selected the members of which pair are complementary to each other with respect to the allelic form of as many of the informative genetic markers as possible, ideally of all informative genetic markers.

Preferably, the complementarity of selected plants is not only assessed at the level of genetic markers, but it is also confirmed phenotypically. When a pair of plants is selected as being substantially complementary and hence suitable to proceed with in the method of the invention, it is advisable to cross these plants with each other (apart from self-pollinating them to give rise to the next generation, and apart from creating DHs from them), to verify whether their progeny displays all or most of the phenotypic characteristics for which the starting organism had been selected. This approach will indicate how far one is still removed from the final goal of the method of this invention (the creation of suitable parental lines to essentially reconstitute the starting organism), because such crosses may provide very useful information about the importance of certain genetic markers in predicting the phenotypic performance of the plant, and which of the genetic markers are actually linked to the superior phenotypic characteristics for which the starting organism had been selected.

Ideally, all informative genetic markers that, when present in a heterozygous form, are correlated to the superior phenotypic characteristics of the starting organism should ultimately be present in a heterozygous form in the offspring of the crossing of the selected parental lines. This is however the ideal scenario, and usually one needs to strive to obtaining a reconstituted hybrid (and its corresponding parental lines) that phenotypically performs equally well or almost as well as the starting organism, while at the same time limiting the costs of the endeavour, in order to keep the procedure economically attractive.

Once genetically complementary plants have been identified the procedure may be stopped and the identified plants can be crossed to each other, to give rise to a hybrid generation that is similar to the partially heterozygous starting organism, with respect to the informative genetic markers that had been used during the procedure and/or with respect to the phenotypic characteristics. The identified complementary plants should preferably have a large degree of homozygosity (ideally they are fully homozygous), so that they give rise to a genetically uniform hybrid, and they can easily be multiplied as a line by self-fertilisation.

The invention thus further relates to a method for reconstructing a partially heterozygous starting organism, comprising:
a) defining a set of genetic markers that are present in a heterozygous form (H) in a partially heterozygous starting organism;
b) producing doubled haploid lines from spores of the starting organism;
c) genetically characterising the doubled haploid lines thus obtained for the said set of genetic markers to determine whether they are present in a first homozygous form (A) or in a second homozygous form (B);
d) selecting at least one pair of doubled haploid lines that have complementary alleles for at least a subset of the genetic markers, wherein each member of the pair is suitable as a parental line for a hybrid organism; and
e) crossing the selected parental lines to give rise to a hybrid organism.

The invention also relates to a method for reconstructing a partially heterozygous starting organism, comprising:
a) defining a set of genetic markers that are present in a heterozygous form (H) in a partially heterozygous starting organism;
b) producing at least one further generation from the starting organism by self-pollination;
c) selecting at least one pair of progeny organisms in which at least one genetic marker from the set is present in a complementary homozygous form (B vs. A, or A vs. B);
d) optionally repeating steps b and c until at least one pair of progeny organisms that have complementary alleles for at least a subset of the genetic markers has been selected as parental lines for a hybrid; and
e) crossing the selected parental lines to give rise to a hybrid organism.

The hybrid organism obtained after crossing parental lines produced according to the invention resembles the partially heterozygous starting organism with respect to at least a subset of the genetic markers. Of course, the marker profile of the new hybrid can also be essentially or completely identical to the marker profile of the starting organism. The wording “a subset of the genetic markers” in steps d) refers to a subset of the genetic markers from the set of genetic markers that had been defined in steps a) of the different methods of the invention. Suitably, the subset of genetic markers referred to in steps d) comprises a majority of the genetic markers from the set of genetic markers, or essentially all genetic markers from the set of genetic markers, or all genetic markers from the set of genetic markers.

In one embodiment, at least a subset of the genetic markers from the set of genetic markers that were present in a heterozygous form in the partially heterozygous starting organism are also present in a heterozygous form in the hybrid organism. This implies that the hybrid organism that results from crossing the selected parental lines is at least partially genetically identical to the starting organism from which it had been derived by means of DH technology or inbreeding, and more specifically, that the hybrid organism scores heterozygous for at least a subset of the genetic markers that scored heterozygous in the starting organism. Preferably, the subset of the genetic markers is genetically linked to at least one desirable phenotypic characteristic of the starting organism, such that said desirable phenotypic characteristic is also present in the hybrid organism that results from crossing the selected parental lines when said genetic marker is scored heterozygous in the hybrid organism.

In a further embodiment, a majority of the genetic markers from the set of genetic markers that were present in a heterozygous form in the partially heterozygous starting organism are also present in a heterozygous form in the hybrid organism. In this embodiment a minority of the genetic markers from the set of genetic markers that were present in a heterozygous form in the partially heterozygous starting organism are thus present in a homozygous form (A or B) in the hybrid organism.

In another embodiment, essentially all genetic markers from the set of genetic markers that were present in a heterozygous form in the partially heterozygous starting organism are also present in a heterozygous form in the hybrid organism. In yet another embodiment, all genetic markers from the set of genetic markers that were present in a heterozygous form in the partially heterozygous starting organism are also present in a heterozygous form in the hybrid organism.

In yet another embodiment, the hybrid organism phenotypically resembles the partially heterozygous starting organism, or the hybrid organism is phenotypically essentially identical to the partially heterozygous starting organism. It is the intention of the method of the invention to reconstitute the genotype of a commercially interesting non-homozygous starting organism, especially with respect to at least one of the phenotypic characteristics that make the starting organism interesting for commercial purposes. Suitably, the hybrid organism is phenotypically identical to the partially heterozygous starting organism, or it is phenotypically superior to the partially heterozygous starting organism due to the absence of one or more undesired traits in the hybrid organism that were present in the starting organism.

The method of this invention can be used with any biological material segregating for genetic markers. The person skilled in the art will recognise that the methods of this invention, although here described for plants, can be applied to biological populations of any organism such as bacteria, yeast, insects, fish, birds, reptiles, amphibians, non-human mammals (such as farm animals) or preferably plant populations.

In one embodiment, the partially heterozygous starting organism is a plant. The present invention can be applied in a broad range of plant species. For example, the invention can be used in species from the genera: Capsicum, Lactuca, Cucumis, Hordeum, Secale, Triticum, Sorghum (e.g., S. bicolor), Zea (e.g., Z. mays), Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Datura, Hyoscyamus, Nicotiana, Solanum (e.g., S. lycopersicum), Petunia, Digitalis, Majorana, Cichorium, Helianthus, Bromus, Asparagus, Antirrhinum, Heterocallis, Nemesis, Pelargonium, Panieum, Pennisetum, Ranunculus, Senecio, Salpiglossis, Browaalia, Glycine, Pisum, Phaseolus, Beta, Citrullus, Cynara, Lolium, Oryza, and Avena. Preferably, the plant species is selected from the group comprising: Solanum, Capsicum, Cucurbita, Cucumis, Brassica, maize, soybean, wheat, canola, sunflower, alfalfa, sorghum, and rice.

The starting point for re-synthesis of parental lines according to the present invention is one particular genotype, arbitrarily designated F1 or “starting hybrid” or “hybrid starting organism” or “partially heterozygous starting organism”. Generally, these terms are intended to refer to an organism that is not fully homozygous, and that thus has a certain degree of heterozygosity in its genome. As such, the genome of this starting organism is at least partially heterozygous.

In one embodiment, this starting organism is suitably an individual plant or a set of plants derived from a vegetatively propagated stock. The latter option is e.g. useful when it is desired to assess the phenotypic performance of the starting organism in more than one location or environmental condition (for which purpose more than a single plant is required), or when selfing of one starting plant does not produce sufficient seeds to perform the method of the invention.

The first step is the production of an F2 population through self-pollination of the starting organism, and in a preferred embodiment the size of the F2 population used for the subject of this invention comprises at least 20 members. A typical population may comprise between about 20 and 200 individuals, but optionally it may comprise 20, 30, 40, 50, 60, 70, 80, 90, 100, 200, 300, 400, 500, 1000, 2500, or 5000 individuals. The size of the population depends on the desired resolution, on the degree of polymorphism within the species and more specifically on the ease or difficulty by which polymorphic genetic markers can be found in the specific F2 population. These parameters are well known for a person skilled in the art. In subsequent cycles of selection, selected F2 plants are self-pollinated to produce F3 populations, selected F3 plants are self-pollinated to produce F4 populations, etcetera, until a satisfactory level of genetic complementarity is obtained in the selected individuals, and crossing of the selected individuals would give rise to a new hybrid that is identical, essentially identical or partially identical to the starting organism, with respect to the informative genetic markers used in the procedure and ideally also with respect to the superior phenotypic characteristics for which the starting organism had been selected.

The number of repeated cycles required to select for complementing plants that are as homozygous as possible depends on different parameters, such as the type of crop, the genome size of the crop, the number of individuals of the successive self-pollinated populations that are being analysed, the recombination frequency, the number of selected genetic markers, and the overall genetic identity that is desired between the hybrid organism and the starting organism. In addition to the methods described above in every cycle of the process (from F2 to F3 and so on), the residual amount of heterozygosity in the respective population can be estimated based on measurements of the amount of heterozygosity of the genetic markers.

In a preferred embodiment individual members of a population derived from a starting organism are selected and genotyped on the basis of the co-dominant scoring of at least 20 genetic markers. For example, a typical population segregates for between about 20 and about 200 genetic markers, but preferably for at least about 20, 30, 40, 50, 60, 70, 80, 90, 100, 200, 300, 400, 500, 1000, 2500, or 5000 genetic markers. The optimal number of genetic markers to be tested depends on the genome size, chromosome number and overall genetic diversity of the species of interest, but generally a saturating number of markers is recommendable to obtain the best results. “Saturating” implies that the vast majority of genomic regions (preferably all genomic regions) is covered by the set of genetic markers, and that essentially all chromosomes and chromosomal regions of the genome may be traced in the offspring with the said set of genetic markers. The co-dominant nature of the genetic markers allows the simultaneous identification of the two allelic forms of each genetic marker, and hence the distinction between homo- or heterozygous presence of the chromosomal regions on which they are located. The genetic markers may include, but are not limited to, marker technologies such as: RFLPs, RAPDs, AFLPs, SSRs, SNPs, indels, and copy-number variants (CNVs).

Genetic markers are specific DNA sequences, genes or gene fragments that may act as chromosomal landmarks. A genetic marker has a known location on a chromosome (with respect to a physical and/or genetic chromosome map), and it preferably co-segregates with a trait or an identifiable phenotypic characteristic of the organism. Genetic markers may thus be used to e.g. follow the inheritance pattern and the presence/absence of a trait, even in developmental stages wherein the phenotypic characteristic itself is not visible, and to determine linkage groups or recombination events. Genetic markers are considered to be informative if at least two different alleles are present in the population under investigation (i.e. if they are polymorphic), and if these different alleles can be conveniently distinguished from each other experimentally.

For the purpose of this invention, informative genetic markers are present in a heterozygous state in the starting organism, i.e. both allelic forms A and B are present in the starting organism. In plants derived from this starting organism (either through DHs made from spores or through self-pollination) these genetic markers will segregate for A and B, and according to the method of the invention individual plants are selected that are complementary for as many of these informative markers as possible, ideally for all informative markers. When crossed to each other, these selected plants will give rise to new hybrid plants in which preferably the majority of the informative markers (ideally all informative markers) are again present in a heterozygous state, such that the new hybrid plants are genetically identical, essentially identical or partially identical to the starting organism, with respect to the informative genetic markers that were used in the procedure.

In a preferred embodiment, the informative genetic markers are known to be linked with desirable phenotypic characteristics in the starting organism. Preferably, the informative genetic markers are linked to phenotypic properties observed in the partially heterozygous starting organism.

In a further preferred embodiment, a reference genome sequence and/or a genetic map of the species of interest is available, and the position of the informative genetic markers on the genome sequence and/or on the genetic map is known. The availability of a genome sequence allows the selection of suitable genetic markers (such as SNPs, indels) in a very informed manner and at the level of individual base pairs, such that an optimal coverage of all chromosomes can be obtained and the homo- or heterozygous presence of essentially all chromosomal regions can be accurately determined in individuals derived from the starting organism by means of DH technology or self-pollination.

If the number of informative genetic markers is increased, this will generally increase the accuracy with which the starting organism can be reconstructed, but it may also make the procedure more laborious. Ideally a good balance should be found between the required efforts (and population size) on the one hand and the completeness of the genetic reconstruction of the starting organism on the other hand. Especially when resources are limited, it is advisable to verify the suitability of pairs of selected plants by crossing them to each other, to be able to assess the phenotypic performance of their hybrid offspring and to determine whether the obtained result is already closely resembling the starting organism, or whether the method of the invention needs to be performed for at least one more round.

RFLPs are the product of allelic differences between DNA restriction fragments caused by nucleotide sequence variability. As is well known to the person skilled in the art, RFLPs are typically detected by extraction of genomic DNA and digestion with a restriction endonuclease. Generally, the resulting fragments are separated according to size and subsequently hybridised with a probe; single copy probes are preferred. Restriction fragments from homologous chromosomes are revealed. Differences in fragment size among alleles represent an RFLP (see, for example, Helentjaris et al., Plant Mol. Biol. 5:109-118 (1985), and U.S. Pat. No. 5,324,631).

In another embodiment, random amplified polymorphic DNA (RAPD) is used as a genetic marker. Random amplified polymorphic DNA or “RAPD” refers to the amplification product(s) between DNA sequences homologous to a single oligonucleotide primer appearing on different sites on opposite strands of DNA. Mutations or rearrangements at or between binding sites will result in polymorphisms as detected by the presence or absence of amplification product and/or length differences of amplified products (see, for example, Welsh and McClelland (1990), Nucleic Acids Res. 18:7213-7218; Hu and Quiros (1991), Plant Cell Rep. 10:505-511).

In yet another embodiment, amplified fragment length polymorphisms (AFLP) are used as a molecular marker. AFLP technology comprises a process that is designed to generate large numbers of randomly distributed molecular markers (see, for example, European Patent EP-0534858).

Simple sequence repeats or “SSR” refers to di-, tri- or tetra-nucleotide tandem repeats within a genome. The repeat region may vary in length between genotypes while the DNA flanking the repeat is conserved such that the same primers will work in a plurality of genotypes. A polymorphism between two genotypes represents repeats of different lengths between the two flanking conserved DNA sequences (see, for example, Akagi et al (1996), Theor. Appl. Genet. 93:1071-1077; Bligh et al. (1995) Euphytica 86:83-85; Struss et al. (1998) Theor. Appl. Genet. 97:308-315; Wu et al. (1993), Mol.Gen.Genet. 241:225-235; U.S. Pat. No. 5,075,217). SSRs are also known as satellites or microsatellites.

Single nucleotide polymorphism or “SNP” refers to a single base pair difference variant sequence (see, for example, Genome Analysis, A Laboratory Manual, E. Green et al., Eds., Volume 4, Cold Spring Harbor Press, Cold Spring Harbor, N.Y., 1989; Ayres et al. (1997), Theor. Appl. Genet. 94:773-781; Landegren et al. (1998), Genome Res. 8:769-776; Wang et al. (1998) Science 280:1077-1082).

Many genetic markers suitable for use with the present invention are publicly available. The person skilled in the art can easily generate suitable genetic markers, as for instance exemplified in: “The DNA Revolution” by Andrew H. Paterson 1996 (Chapter 2) in: “Genome Mapping in Plants” (ed. Andrew H. Paterson) by Academic Press/R. G. Landis Company, Austin, Tex., pp. 7-21).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will be further illustrated in the examples described hereafter, which examples are not intended to be limiting in any way. In this application and the examples reference is made to the following figures:

FIG. 1 illustrates the progressive fixation of genetic markers in the F2, F3, F4, F5 and F6 generations, derived from a partially heterozygous starting organism (F1) through repeated selfing. In each generation panel the marker scores for a number of individuals are presented. Each horizontal line corresponds to a different individual, and from left to right the different linkage groups are represented. The allelic form of each marker is indicated in colour coding: red (dark grey)=homozygous “allele A” genotype, blue (black)=homozygous “allele B” genotype, green (light grey)=heterozygous “H”.

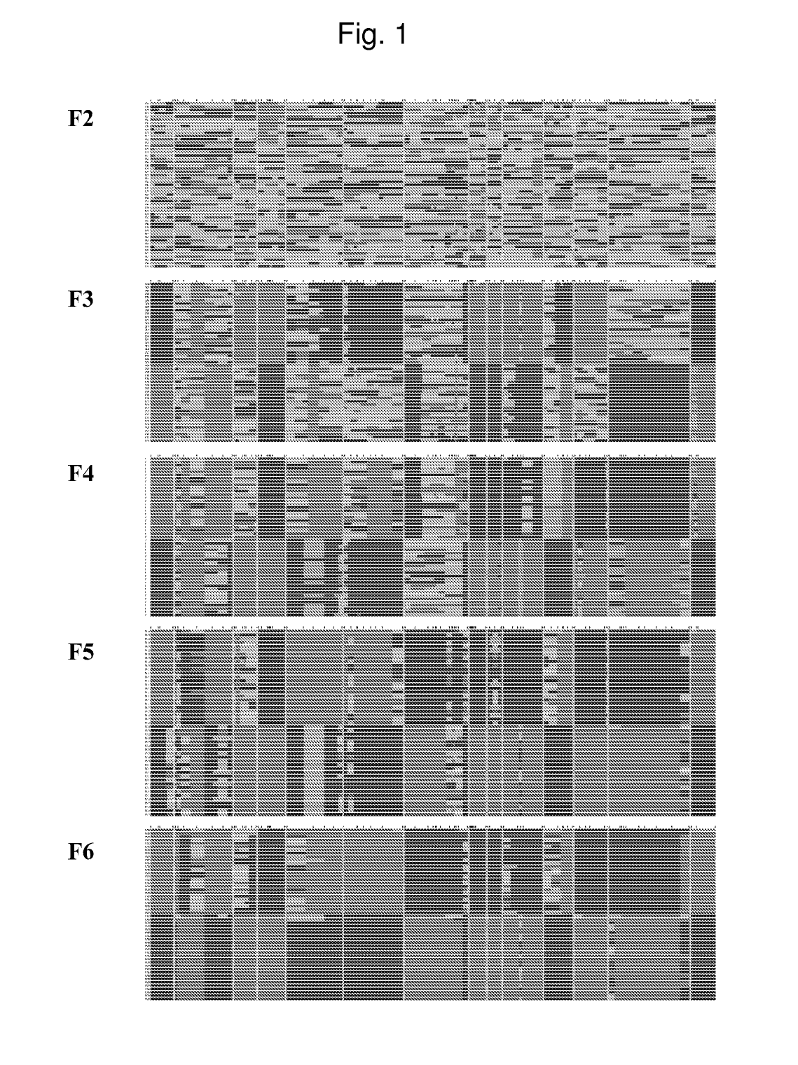

FIG. 2A presents an overview of the genotypes of 90 individuals of the F2 population of Example 1. Each horizontal line represents an individual. Each vertical block represents a linkage group (total of 19 linkage groups). Red (dark grey)=homozygous “allele A” genotype, blue (black)=homozygous “allele B” genotype, green (light grey)=heterozygous “H”. The black bars above the illustration designate the genetic marker positions.

FIGS. 2B and 2C present the genotypes of the two pairs of selected F2 plants of Example 1.

### EXAMPLES

### Example 1

A tomato cultivar excelling in its high productivity and exceptionally good taste was first found as an individual plant in a segregating population in breeding trials. It was however recognised that in this stage of breeding the genetic composition of the plant was complex, in the sense that the traits for which the individual plant was appreciated would not breed true. Therefore, it was not possible to produce parental lines from this individual plant that would, when crossed with each other, give rise to a hybrid that resembles its unique genetic composition and that hence would allow its commercialisation in the traditional way by seeds. The only way this superior plant could be commercialised was by vegetative propagation. This invention teaches the methodology that allowed the generation of a similar genotype and phenotype in a true seed form.

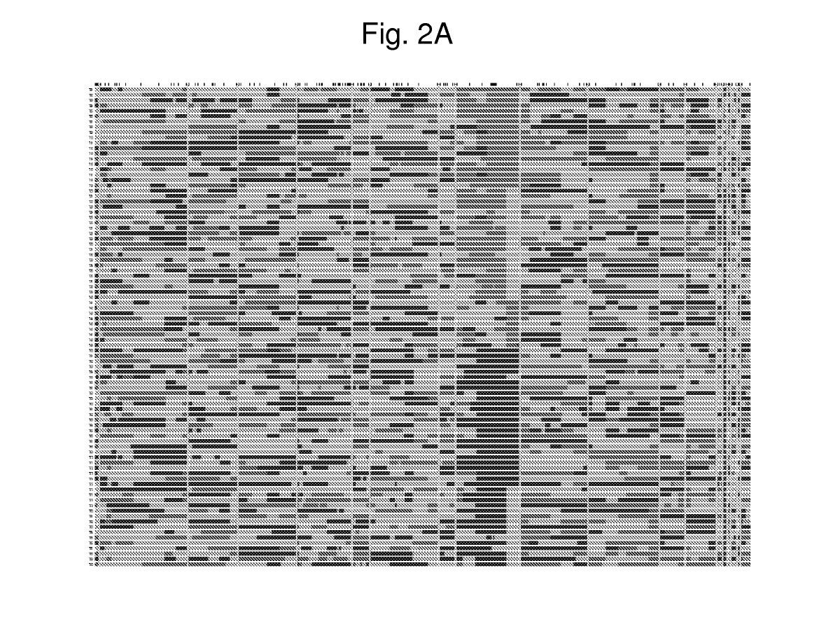

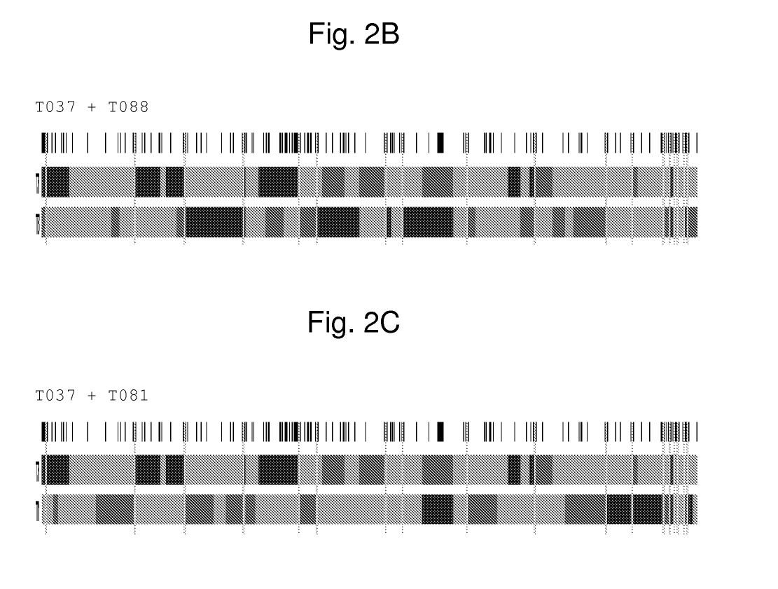

Clonally propagated plants of the tomato cultivar were grown in the greenhouse in De Lier, The Netherlands. Seeds from one individual plant (the partially heterozygous starting organism, F1), which was self-pollinated, were harvested and sown out (=F2 population). Ninety-six individual F2 plants were retained and DNA analysis was performed. An initial pre-screening on a random subset of the 96 individual F2 plants resulted in the selection of a set of polymorphic genetic markers that could be scored co-dominantly (FIG. 2A). Analysis of the total set of genetic markers on the 96 individual F2 plants yielded a total of 156 polymorphic genetic markers. These 156 informative genetic markers could be assembled into 19 linkage groups using a commercially available software package (see, for example, JoinMap®; http://www.kyazma.nl/index.php/mc.JoinMap/).

Based on the genetic marker scores for the individual F2 plants and their corresponding graphical genotype displays, two pairs of plants were selected that complemented each other for a subset of the genetic markers (i.e. for a considerable number of the genetic markers the two plants of each individual pair scored A in one of the selected plants and that scored B in the complementary plant of the pair). The remaining genetic markers were only fixed for one allele (A or B) in one of the selected plants, whereas in the complementary plant these markers were heterozygous (H), thus still allowing for the selection of plants fixed for the other allele (B or A) in subsequent generation(s). FIGS. 2B and 2C illustrate the degree of genetic complementarity of the selected F2 plants. The originally selected sets of plants were self-pollinated to produce F3 populations. Depending on the degree of heterozygosity of the selected sets of plants, an F3 population is sufficient for obtaining parental lines that are true breeding. In other cases, F4 or even F5 or F6 populations may be required.

In this case, two pairs of F4 plants were eventually selected as suitable parents for new hybrid plants that were genetically very similar to the starting organism, with respect to the used set of genetic markers. When crossed, each pair of selected plants gave rise to a population of hybrid plants that also phenotypically resembled the starting organism very well, and in this manner suitable parental lines (i.e. true breeding for the relevant characteristics) were made for this tomato variety, allowing it to be reproduced through seeds, rather than vegetatively. The selected pairs of parental lines were sufficiently homozygous to be sexually multiplied as a line by means of self-pollination.

### Example 2

A phenotypically superior plant was identified in a segregating cucumber breeding population. To allow the rapid, large-scale and reproducible sexual propagation of this partially heterozygous cucumber plant, there was a need to create suitable parental lines that could be crossed to give rise to hybrid plants that were phenotypically identical or very similar to the starting plant.

The complete genomic sequence of the starting plant was determined by means of Illumina sequencing, and based on this sequence a set of informative genetic markers was selected. The selection comprised 87 genetic markers for which the starting plant was heterozygous, such that the homo- or heterozygosity of all seven chromosome pairs could be queried with the set of genetic markers, and the genetic markers were evenly spaced across each of the chromosomes.

Subsequently, a population of DHs was created from the starting plant by means of gynogenesis by means of the gynogenesis protocol for Cucumis sativus as described in European Patent EP-0374755.

From among this population 210 DHs were tested for the selected set of genetic markers, and the results were processed and compared by means of suitable software. On the basis of the resulting genetic marker profiles for each DH line, three pairs of DHs were identified and selected. The two individuals within each pair of DHs were pairwise complementary for the majority of the tested genetic markers. The advantage of using DHs in the method of the invention is that all genetic markers are per definition present in a homozygous form (i.e. 100% homozygosity), and in contrast to the situation in Example 1 no additional generations were thus required to further increase the overall homozygosity. The disadvantage of using DHs in the method of the invention is that all alleles are immediately fixed in one of the two allelic forms, and that no further segregation can take place in subsequent generations.

The selected DHs of each pair were crossed to each other to give rise to a hybrid generation, which was subsequently phenotypically compared to the starting plant. This finally led to the selection of one pair of DHs that, when crossed, gave rise to a hybrid that most closely resembled the phenotypically superior qualities of the starting plant. In addition, an undesirable phenotypic characteristic that had been present in the starting plant was absent from the selected DHs, such that the hybrid resulting from crossing these DHs did not have this undesirable phenotypic characteristic. In this manner, suitable parental lines were created for the superior hybrid plant, without a need for sexual propagation of the hybrid which would lead to the segregation of all traits.

### Example 3

Using the cucumber plant of Example 2 as the partially heterozygous starting plant, a different approach was also followed, in parallel to the procedure outlined in Example 2. In this parallel approach, the superior cucumber plant was self-pollinated, and a resulting F2 population of 24 individuals was screened for the same set of 87 informative SNP markers. One F2 plant was selected based on its high degree of complementarity to one of the six selected DHs mentioned in Example 2, while being heterozygous for nearly all of the remaining SNPs for which there was no complementarity.

This situation was interesting because the allelic fixation in this F2 plant was nearly completely limited to alleles that were fixed in the complementary form in the selected DH line, whereas the SNPs that were present in a heterozygous state in the F2 plant retained the potential to segregate for both co-dominant alleles in the F3 generation (derived from selfing the F2 plant), thus allowing the identification and selection of F3 plants that were complementary with the selected DH plant for an even larger number of the SNPs. The selected F2 plant was self-pollinated to give rise to an F3 population, and the method of the invention was further applied until the F4 generation. From among an F4 population 50 plants were genetically analysed for the set of 87 genetic markers, and one individual was identified that was almost entirely complementary to the selected DH, with respect to the said set of markers.

This F4 individual was subsequently crossed with the previously selected DH line (which had been propagated by means of selfing), to give rise to a progeny population that uniformly displayed the superior characteristics of the starting plant. Thus suitable parental lines were created to allow for the reproducible sexual propagation of this starting plant.

1. Method for producing parental lines for a hybrid organism, comprising:
a) defining a set of genetic markers that are present in a heterozygous form (H) in a partially heterozygous starting organism;
b) producing doubled haploid lines from spores of the starting organism;
c) genetically characterising the doubled haploid lines thus obtained for the said set of genetic markers to determine whether they are present in a first homozygous form (A) or in a second homozygous form (B);
d) selecting at least one pair of doubled haploid lines that have complementary alleles for at least a subset of the genetic markers, wherein each member of the pair is suitable as a parental line for a hybrid organism.

2. Method for producing parental lines for a hybrid organism, comprising:
a) defining a set of genetic markers that are present in a heterozygous form (H) in a partially heterozygous starting organism;
b) producing at least one further generation from the starting organism by self-pollination;
c) selecting at least one pair of progeny organisms in which at least one genetic marker from the set is present in a complementary homozygous form (B vs. A, or A vs. B);
d) optionally repeating steps b) and c) until at least one pair of progeny organisms that have complementary alleles for at least a subset of the genetic markers has been selected as parental lines for a hybrid.

3. Method as claimed in claim 1 or 2, wherein the genetic markers from the set of genetic markers are distributed essentially evenly across the genome and/or across the genetic map of the organism.

4. Method as claimed in claim 1 or 3, wherein the subset of genetic markers comprises a majority of the genetic markers from the set of genetic markers, or essentially all genetic markers from the set of genetic markers, or all genetic markers from the set of genetic markers.

5. Method for reconstructing a partially heterozygous starting organism, comprising the method of any one of the claims 1-4 and further comprising the step of crossing the selected parental lines to give rise to a hybrid organism.

6. Method as claimed in claim 5, wherein at least a subset of the genetic markers from the set of genetic markers that were present in a heterozygous form in the partially heterozygous starting organism are also present in a heterozygous form in the hybrid organism.

7. Method as claimed in claim 5 or 6, wherein a majority of the genetic markers from the set of genetic markers, or essentially all genetic markers from the set of genetic markers, or all genetic markers from the set of genetic markers that were present in a heterozygous form in the partially heterozygous starting organism are also present in a heterozygous form in the hybrid organism.

8. Method as claimed in any one of the claims 5-7, wherein the hybrid organism phenotypically resembles the partially heterozygous starting organism, or wherein the hybrid organism is phenotypically essentially identical to the partially heterozygous starting organism.

9. Method as claimed in claim 1 or 2, wherein the doubled haploid lines of step b) are produced in vitro by means of gynogenesis or androgenesis, followed by diploidisation, in particular spontaneous or chemical-induced diploidisation.

10. Method as claimed in claim 1 or 2, wherein the doubled haploid lines are produced in vivo, in particular by means of a haploid-inducer system.

11. Method as claimed in any one of the claims 1-10, wherein the genetic markers are selected from the group comprising RFLPs, RAPDs, AFLPs, SSRs, SNPs, indels, copy-number variants.

12. Method as claimed in any one of the claims 1-11, wherein the number of genetic markers is at least about 20, 30, 40, 50, 60, 70, 80, 90, 100, 200, 300, 400, 500, 1000, 2500, or 5000.

13. Method as claimed in any one of the claims 1-12, wherein the partially heterozygous starting organism is a plant.

14. Method as claimed in claim 13, wherein the partially heterozygous starting organism belongs to a species selected from the group comprising maize, rice, potato, wheat, barley, cotton, soybean, canola, tomato, cucumber, pepper, watermelon, melon, carrot, bean, pea, sorghum, sunflower, fennel, cauliflower, lettuce, head cabbage, broccoli, chicory, asparagus, endive, radish, chickpea, spinach.
